(19) United States
(12) Patent Application Publication
(10) Pub. No.: US 2020/0154992 A1
(43) Pub. Date: May 21, 2020

(54) BUTTON ADAPTER FOR DISPOSABLE URETEROSCOPE

(71) Applicant: BOSTON SCIENTIFIC SCIMED, INC., Maple Grove, MN (US)

(72) Inventors: Thomas Michael ZAPPIA, JR., West Boylston, MA (US); Niraj Prasad RAUNIYAR, Plymouth, MN (US); Roy BROMANDER, Salem, NH (US)

(21) Appl. No.: 16/553,401

(22) Filed: Aug. 28, 2019

Related U.S. Application Data
(60) Provisional application No. 62/768,271, filed on Nov. 16, 2018.

(51) Int. Cl. A61B 1/307; A61B 1/00

(52) U.S. Cl. CPC A61B 1/307; A61B 1/00066; A61B 1/0014; A61B 1/00121

(57) ABSTRACT

An adapter for a disposable medical scope device includes a body configured to be removably attached to a scope device. The adapter includes at least one button including an actuator configured to activate at least one circuit within a scope device to which it is attached. The body is configured to be electrically coupled to a control unit such that, when the circuit is activated by the button, a signal is sent from the adapter to the control unit to control the control unit's handling of output from the scope device.

### PRIORITY CLAIM

The present disclosure claims priority to U.S. Provisional Patent Application Ser. No. 62/768,271 filed Nov. 16, 2018; the disclosure of which is incorporated herewith by reference.

### FIELD

Aspects of the present disclosure generally relate to medical devices and methods. Particular aspects relate generally to endoscopic devices and methods.

### BACKGROUND

Programmable buttons on endoscopes have been used to control features, such as image capture, white balance, illumination control, etc. These buttons have been incorporated into the device, usually somewhere on the handle, and control these endoscope features with direct communication to a control unit connected to the endoscope.

For disposable endoscopes, it is beneficial to minimize features to reduce component cost. Buttons added to the disposable endoscopes can control similar features but need to be programmed to interface with the control unit. Additional features cannot be added without updating the control unit or adding interface communication to other equipment, such as laser control, video recording or fluid management. Thus, there is a need for an adapter that may be optionally connected to a disposable endoscope and contains buttons that can control various features without updates or connections to the control unit.

### SUMMARY

The present disclosure relates to an adapter for a disposable medical scope device comprising a body configured to be removably attached to a scope device, the adapter including at least one button including an actuator configured to activate at least one circuit within a scope device to which it is attached, the body configured to be electrically coupled to a control unit such that, when the circuit is activated by the button, a signal is sent from the adapter to the control unit to control the control unit's handling of output from the scope device.

In an embodiment, a circuit switch of the at least one circuit is indirectly actuated by the button, preventing a fluid pathway into the scope device.

In an embodiment, the button is one of a magnet or conductive material.

In an embodiment, the adapter includes mating features configured to couple the adapter to the scope device.

In an embodiment, the mating features are clips.

In an embodiment, the at least one button is configured to interface with an electrical contact pad on the scope device.

In an embodiment, the control unit includes an image display, the adapter being configured to control display features of the image display when the adapter is coupled to the scope device.

In an embodiment, the display features include white balance, image capture and brightness.

The present disclosure also relates to an adapter for a disposable medical scope device comprising a body configured to be removably attached to a scope device, the adapter including at least one button including an actuator configured to activate at least one circuit within a scope device to which it is attached, the body configured to be electrically coupled to at least one external system such that, when the circuit is activated by the button, a signal is sent from the adapter to the external system to control the external system's output to the scope device.

In an embodiment, the body is configured to be electrically coupled to first and second external systems.

In an embodiment, the body is configured to be wirelessly electrically coupled to the first and second external systems.

In an embodiment, the body is configured to be directly electrically coupled to the first and second external systems via a control unit such that the adapter controls the first and second external systems via signals sent through the control unit.

In an embodiment, the buttons of the adapter are programmed with a first sequence code corresponding to the first external system and a second sequence code corresponding to the second external system, each of the first and second sequence codes sending a signal to the control unit to adjust settings of the corresponding external system when the adapter is coupled to the scope device.

In an embodiment, the first external system is a laser system and the second external system is a fluid management system.

In an embodiment, a circuit switch of the at least one circuit is indirectly actuated by the button, preventing a fluid pathway into the scope device.

The present disclosure also relates to a method of controlling a scope system from a scope device comprising positioning an adaptor over a scope device, the scope device being electrically connected to a control unit, the adaptor comprising: a body configured to be removably attached to a scope device, the adapter including at least one button including an actuator configured to activate at least one circuit within a scope device to which it is attached, the body configured to be electrically coupled to at least one external system such that, when the circuit is activated by the button, a signal is sent from the adapter to the external system to control the external system's output to the scope device, and actuating the button to control features of the control unit and the external system from the scope device.

In an embodiment, a circuit switch of the at least one circuit is indirectly actuated by the button, preventing a fluid pathway into the scope device.

In an embodiment, the button is one of a magnet or conductive material.

In an embodiment, the adapter includes mating features configured to couple the adapter to the scope device.

In an embodiment, the at least one button is configured to interface with an electrical contact pad on the scope device.

### BRIEF DESCRIPTION

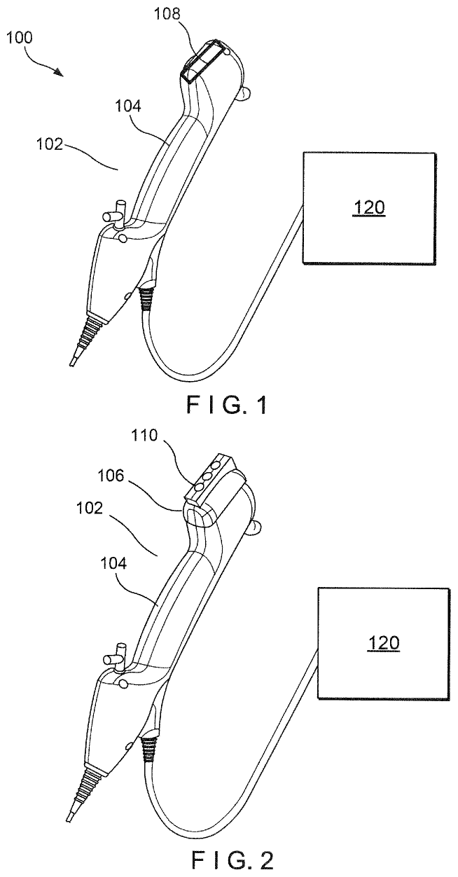

FIG. 1 shows a perspective view of a scope device according to an exemplary embodiment of the present disclosure;

FIG. 2 shows a perspective view of a scope device system according to an exemplary embodiment of the present disclosure;

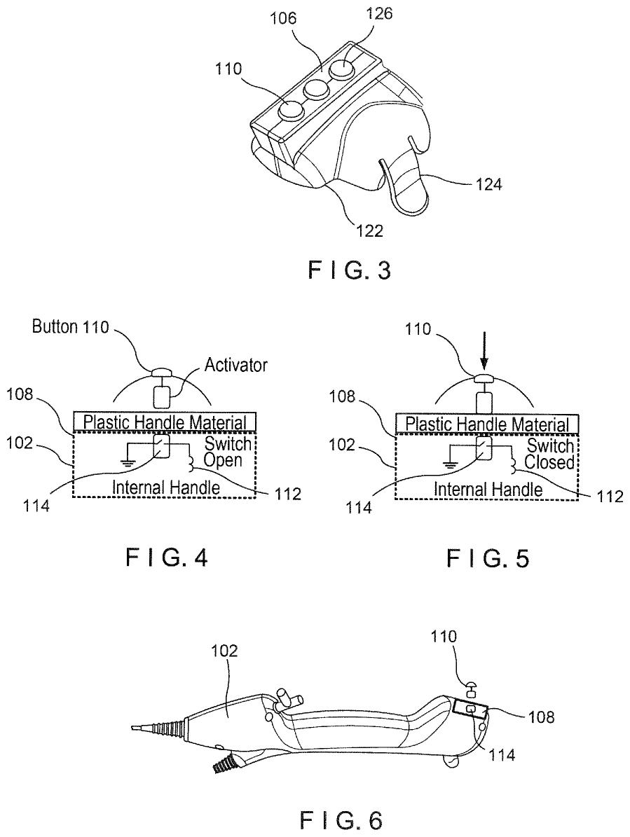

FIG. 3 shows a perspective view of an adapter of the scope device system of FIG. 2;

FIG. 4 shows an internal view of the electrical components of the scope device and adapter of the scope device system of FIG. 2 according to an exemplary embodiment of the present disclosure;

FIG. 5 shows another internal view of the electrical components of the scope device and adapter of the scope device system of FIG. 2;

FIG. 6 shows a perspective view of the scope device of FIG. 1;

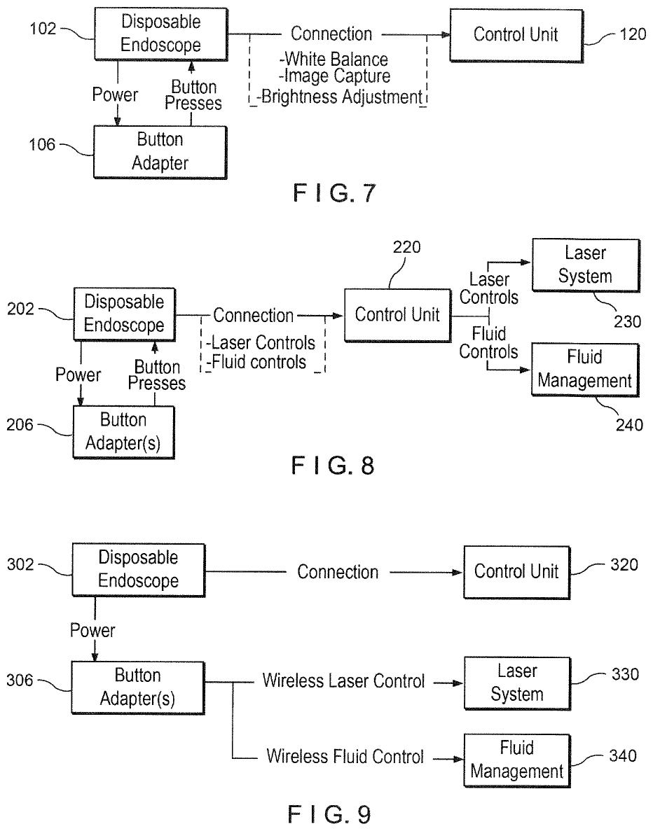

FIG. 7 shows a schematic diagram of the interfaces between the components of the scope device system of FIG. 1 according to a first exemplary embodiment of the present disclosure;

FIG. 8 shows a schematic diagram of the interfaces between the components of the scope device system of FIG. 1 according to a second exemplary embodiment of the present disclosure; and

FIG. 9 shows a schematic diagram of the interfaces between the components of the scope device system of FIG. 1 according to a third exemplary embodiment of the present disclosure.

### DETAILED DESCRIPTION

The present disclosure may be further understood with reference to the following description and appended drawings, wherein like elements are referred to with the same reference numerals. The present disclosure relates, for example, to devices, systems and methods for the removal of debris within a lumen (e.g., within a living body). Exemplary embodiments describe a system including an adapter optionally connected to a disposable endoscope or ureteroscope devices, such as the LithoVue™, which contains buttons that can control various features of the scope device without updates or connections to the control unit. In some embodiments, the adapter may be electrically interfaced with the scope device to control the various features. In other embodiments, the adapter may wirelessly control the various features from the scope device. It should be noted that the terms “proximal” and “distal”, as used herein, are intended to refer to a direction toward (proximal) and away from (distal) a user of the device (e.g., physician).

As shown in FIGS. 1-6, a scope system 100 according to an exemplary embodiment of the present disclosure comprises, in an exemplary embodiment, a disposable scope device 102 including a handle 104 which, during use, remains outside a living body, an adapter 106 and a control unit 120. As shown in FIG. 1, the scope device 102 may comprise any scope configured for use in minimally invasive procedures, such as a ureteroscope, for example, under the brand name LithoVue™ or Next Generation LithoVue™. The scope device 102 is connected via, for example, a plug, to a control unit 120, such as a computer, with an image display. The control unit 120 provides power to the scope device 120 and allows the adapter 106 to control various functions of the scope system 100. The control unit 120 is also operatively connected to any external equipment or systems such as, for example, a laser system, a fluid management system and/or image/video capture devices. Thus, the control unit 120 is capable of controlling various functions of the connected systems and devices. In order for the adapter 106 to control various features of the scope system 100, the scope device 102 includes an electrical contact pad 108 positioned thereon that is configured to interface with electrical components of the adapter 106. The contact pad 108 provides power from the control unit 120 to the adapter 106. The contact pad 108 also uses simple electronics to provide an electrical connection between the scope device 102 and a plurality of buttons 110 on the adapter 106. For example, as shown in FIGS. 4-5, the handle 104 may be internally hardwired with at least one circuit 112 with a switch 114 that may be activated (i.e., opened or closed) when a button 110 on the adapter 106 is pressed. The circuit 112, when closed, is hardwired, in this embodiment, to send a signal through the scope device 102 to the control unit 120. It is noted that although FIGS. 4-5 show a single circuit 112 and button 110, any number of circuits may be connected to the contact pad 108 to allow for adaptability of the adapter 106 depending on the procedure and number of systems connected to the control unit 120. In an exemplary embodiment, the circuit switch 114 is indirectly controlled by the button 110 (i.e., through used of a magnet, conductive material, etc.), preventing a fluid pathway into the scope device 102. That is, the contact pad 108 is waterproof with all electrical components being protected from external fluids via, for example, a layer of plastic handle material.

The adapter 106 is configured to be removably attached to a portion of the handle 104 of the scope device 102. As shown in FIG. 2 the adapter 106, in this embodiment, is attachable to a proximal end of the scope device 102. However, it will be understood by those skilled in the art that the adapter 106 may be attachable to any portion of the scope device 102, depending on the preferences of the physician performing the procedure. The adapter 106 is, in this embodiment, a molded component formed of, for example, plastic. The adapter 106 may be molded so that a first surface 122 thereof (i.e., a surface that interfaces with an outer surface of the scope device 102) form fits against a specific contour of the outer surface of the handle 104, providing a low-profile addition to the scope handle 104. Mating features 124 (i.e., clips) extend from lateral sides of the adapter 106 and may clip into receiving portions (not shown) of the handle 104 to provide a more secure fit between the adapter 106 and the handle 104. It will be understood by those skilled in the art that although clips are shown in the present embodiment, any mating features may be used to couple the adapter 106 to the handle 104 such as, for example snap-fits or magnetic connectors. A plurality of buttons 110 are incorporated in the adaptor 106 and are configured to interface with the electrical contact pad 108 on the scope device 102. It will be understood that although FIGS. 2-3 depicts three buttons 110, any number of buttons 110 may be incorporated into the adapter 106, depending on the requirements of the procedure and/or physician. Each button 110 consists of an actuator such as, for example, a magnet used in a reed switch or a conductive material used in a capacitive switch, which causes the circuit 112 in the handle 104 to open or close, sending a button press signal through the scope device 102 to the control unit 120. Specifically, as each button 110 is pressed, a surface 126 of the button 110 closest to the handle 104 abuts the plastic material of the handle 104 covering the contact pad 108. This proximity of the pressed button 110 to the circuit 112 is enough to actuate the circuit switch 114. The button 110, in this embodiment, does not directly interact with the circuit 112, as noted above, to prevent a fluid pathway into the scope device 102 and allowing the adapter 106 to be an optional component. Thus, because the adapter 106 is an optional component that can interface with the handle 104 as desired, the adapter 106 can expand the use and addition of external features to the scope system 100 without needing to incorporate a control design into the scope device 102. Furthermore, the adapter 106 allows expansion of feature usage of disposable ureteroscopes while minimizing cost and offering customers a wider range of features with other equipment such as laser systems, fluid management systems and image/video capture systems.

FIG. 7 depicts an exemplary embodiment of the electrical connections and control features between the scope device 102, the adapter 106 and the control unit 120 of an exemplary scope system 100. In this embodiment, the adapter 106 is capable of controlling an image display of the control unit 120 through its connection with the scope device 120 when positioned on the handle 104. In an exemplary embodiment, each button may control a different aspect of the image display. For example, a first button may control the white balance of the image display while a second button controls image capture and a third button controls the brightness of the image display. It will be understood, however, that the buttons 110 may be programmed to control any features of the display and are not limited to the above-identified features. Furthermore, the adapter 106 may include any number of buttons 110 depending on the procedure or the preferences of the user.

Looking to FIG. 7, the control unit 120 is the source of power to the scope device, which provides power to the adapter 106 when the two components interface at the contact pad 108. The adapter 106 may be pre-programmed to then control the display features through its connection to the scope device 100 and the connection of the scope device 100 to the control unit 120.

FIG. 8 depicts another exemplary embodiment of the electrical connections and control features between the scope device 202, the adapter 206, the control unit 220, and two external systems—a laser system 230 and a fluid management system 240. Each of the laser system 230 and the fluid management system 240 is electrically connected to the control unit 220 via, for example, a direct line such as a plug. In this embodiment, the adapter 206 is capable of controlling the laser system 230 and the fluid management system 240 when positioned on the handle 204. In an exemplary embodiment, specific signal codes may be pre-assigned to the laser system 230 and the fluid management system 240, via the control system 220, which allow the adapter 206 to control these features beyond the control unit 220. For example, if the laser system 230 is connected to the control unit 220, a certain code sequence may be entered using the buttons 210 on the adapter 206. This sequence is sent through the control unit 220 to the laser system 230 to adjust laser settings or use. If the fluid management system 240 is connected to the control unit 220, a different code sequence may be entered using the buttons 210, sending a signal through the control unit to the fluid management system 240 to adjust the fluid management system settings. In another exemplary embodiment, rather than using sequence codes, two separate adapters 206 may be used, a first adapter 206 for the laser system 230 and a second adapter 206 for the fluid management system 240. Because the adapters 206 are removable, when the user wants to control, for example, the laser system 230, the first adapter 206 is simply coupled to the handle 204. If, during the procedure, the user wants to switch to controlling the fluid management system 240, the user need only remove the first adapter 206 from the handle 204 and couple the second adapter 206, which is programmed to the fluid management system 240, to the handle 204.

Looking to FIG. 8, the control unit 220 is the source of power to the scope device 202, which provides power to the adapter 206 when the two components interface. Following the arrows from the adapter 206, once the adapter 206 is connected to the scope device 202, button presses activate the circuit in the disposable endoscope which, through the connection to the control unit 220, controls the laser system 230 and/or the fluid management system 240.

FIG. 9 depicts a scope system 300 according to another exemplary embodiment of the present disclosure. The system 300, in this embodiment, also includes a scope device 302, an adapter 306, a control unit 320, and two external systems—a laser system 330 and a fluid management system 340. However, in this embodiment, the laser system 230 and the fluid management system 340 are wirelessly controlled by the adapter 306. In this embodiment, the control unit 320 is the source of power to the scope device 302, which provides power to the adapter 306 when the two components interface. Specifically, the scope device 302 is electrically connected to the control unit 320 via, for example, a direct line such as a plug. The laser system 330 and the fluid management system 340, however, are separate components that are not directly electrically connected to the control unit 320. Thus, these systems may receive power from other sources (i.e., an electrical outlet), Once connected to the scope device 300, the adapter 306 is able to send control signals to the laser system 330 and the fluid management system 340 with, for example, short wave radio frequencies. As with the scope device 200, specific signal codes may be pre-assigned to the laser system 330 and the fluid management system 340 which allow the adapter 306 to control these features. In another exemplary embodiment, rather than using sequence codes, two separate adapters 306 may be used, a first adapter 306 for the laser system 330 and a second adapter 306 for the fluid management system 340. Because the adapters 306 are removable, when the user wants to control, for example, the laser system 330, the first adapter 306 is simply coupled to the handle 304. If, during the procedure, the user wants to switch to controlling the fluid management system 340, the user need only remove the first adapter 306 from the handle 304 and couple the second adapter 306, which is programmed to the fluid management system 340, to the handle 304. Thus, this embodiment allows for compatibility with future features and systems that are not on the market when the endoscope is designed.

It will be appreciated by those skilled in the art that changes may be made to the embodiments described above without departing from the inventive concept thereof. It should further be appreciated that structural features and methods associated with one of the embodiments can be incorporated into other embodiments. It is understood, therefore, that this invention is not limited to the particular embodiment disclosed, but rather modifications are also covered within the scope of the present invention as defined by the appended claims.

1-15. (canceled)

16. An adapter for a disposable medical scope device, comprising:
a body configured to be removably attached to a scope device, the adapter including at least one button including an actuator configured to activate at least one circuit within a scope device to which it is attached, the body configured to be electrically coupled to a control unit such that, when the circuit is activated by the button, a signal is sent from the adapter to the control unit to control the control unit's handling of output from the scope device.

17. The system of claim 16, wherein the circuit switch is indirectly actuated by the button, preventing a fluid pathway into the scope device.

18. The system of claim 16, wherein the button is one of a magnet or conductive material.

19. The system of claim 16, wherein the adapter includes mating features configured to couple the adapter to the scope device.

20. The system of claim 19, wherein the mating features are clips.

21. The system of claim 16, wherein the scope device is a disposable scope device.

22. The system of claim 16, wherein the control unit includes an image display, the adapter being configured to control display features of the image display when the adapter is coupled to the scope device.

23. The system of claim 22, wherein the display features include white balance, image capture and brightness.

24. An adapter for a disposable medical scope device, comprising:
a body configured to be removably attached to a scope device, the adapter including at least one button including an actuator configured to activate at least one circuit within a scope device to which it is attached, the body configured to be electrically coupled to at least one external system such that, when the circuit is activated by the button, a signal is sent from the adapter to the external system to control the external system's output to the scope device.

25. The adapter of claim 24, wherein the body is configured to be electrically coupled to first and second external systems.

26. The adapter of claim 25, wherein the body is configured to be wirelessly electrically coupled to the first and second external systems.

27. The adapter of claim 25, wherein the body is configured to be directly electrically coupled to the first and second external systems via a control unit such that the adapter controls the first and second external systems via signals sent through the control unit.

28. The adapter of claim 25, wherein the buttons of the adapter are programmed with a first sequence code corresponding to the first external system and a second sequence code corresponding to the second external system, each of the first and second sequence codes sending a signal to the control unit to adjust settings of the corresponding external system when the adapter is coupled to the scope device.

29. The adapter of claim 25, wherein the first external system is a laser system and the second external system is a fluid management system.

30. The adapter of claim 24, wherein a circuit switch of the at least one circuit is indirectly actuated by the button, preventing a fluid pathway into the scope device.

31. A method for controlling a scope system from a scope device, comprising:
positioning an adaptor over a scope device, the scope device being electrically connected to a control unit, the adaptor comprising:
a body configured to be removably attached to a scope device, the adapter including at least one button including an actuator configured to activate at least one circuit within a scope device to which it is attached, the body configured to be electrically coupled to at least one external system such that, when the circuit is activated by the button, a signal is sent from the adapter to the external system to control the external system's output to the scope device; and
actuating the button to control features of the control unit and the external system from the scope device.

32. The device of claim 31, wherein a circuit switch of the at least one circuit is indirectly actuated by the button, preventing a fluid pathway into the scope device.

33. The device of claim 31, wherein the button is one of a magnet or conductive material.

34. The device of claim 31, wherein the adapter includes mating features configured to couple the adapter to the scope device.

35. The device of claim 31, wherein the at least one button is configured to interface with an electrical contact pad on the scope device.
